(19) United States
(12) Patent Application Publication
(10) Pub. No.: US 2007/0276331 A1
(43) Pub. Date: Nov. 29, 2007

(54) Device for retaining the external portion of a peripherally inserted central catheter

(76) Inventor: Helen Campbell, Columbus, OH (US)

Correspondence Address: TAFT, STETTINIUS & HOLLISTER LLP, SUITE 1800, 425 WALNUT STREET, CINCINNATI, OH, 45202-3957, US

(21) Appl. No.: 11/805,255

(22) Filed: May 22, 2007

Related U.S. Application Data
(60) Provisional application No. 60/808,211, filed on May 24, 2006.

(51) Int. Cl. A61M 5/32

(52) U.S. Cl. 604/174

(57) ABSTRACT

A storage and support device, comprising a band with an external pocket for retaining on a patient's body the external portion of an infusion device in a position approximate to the exit site of the infusion device. Such storage and support device may be made from a sheet material and may be a fixed circular band, or a rectangular strip of sheet material with fastening means along the opposite sides, such that the strip becomes a band when the two sets of fastening means are engaged. A biasing means may be used with any embodiment of the present invention, to aid in retention of the storage and support device in the proper position on the patient's body. More specifically, the present invention may be adapted be worn on a patient's arm to retain the external portion of a peripherally inserted central catheter.

### CROSS REFERENCE TO RELATED APPLICATIONS

This application claims the benefit of U.S. Provisional Patent Application Ser. No. 60/808,211, entitled “Device for Retaining the External Portion of a Peripherally Inserted Central Catheter”, filed on May 24, 2006, the disclosure of which is incorporated herein by reference.

### BACKGROUND

1. Field of the Invention

The present invention relates to healthcare products. More specifically, it relates to storage and support devices that can be worn by a patient to retain the external portion of an infusion device on the patient's body, approximate to the exit site of the infusion device. The healthcare industry would benefit from this invention.

2. Background of the Invention

The present invention provides a device for retaining the external portion of an infusion device, such as a peripherally inserted central catheter.

A peripherally inserted central catheter (hereinafter a “PICC” or “PICC line”) is an example of an infusion device that has been adapted for long-term use. A PICC includes a flexible tube, or catheter, that is inserted into a vein in a patient's arm, and threaded through the vein to a termination point under the patient's clavicle, close to the heart. A PICC may be used for administering fluids, medication, nutrients, and/or blood products as well as for drawing blood samples. In contrast to a regular intravenous line that may remain in place for only a matter of days, a single PICC line may remain in a patient's arm for months, provided it is maintained properly. This makes PICC lines ideal for routine administration of medications over extended periods of time, as may be required for chemotherapy and many long-term antibiotic therapies.

A PICC line is usually anchored at the exit site (i.e. the place on the patient's arm where the PICC enters the vein) by one or more adhesive bandages, such as Steri-Strips™ and/or a piece of Tegaderm™. This leaves some section of loose tubing, terminating in a PRN adaptor or other type of adaptor, extending from the exit site dressing. While the exit site dressing is changed at least once per week, this external portion of the PICC line often must be accessed more frequently to facilitate administration of medications and/or blood draws. At the same time, however, it is important that the external portion not be permitted to simply hang free, as excessive disturbance may cause movement of the internal portion of the PICC line, a kink in the PICC line, damage to the PICC line, pain or irritation, mechanical phlebitis, or even infection.

Traditionally, the challenge of sufficiently anchoring the external portion of long-term use catheter, while allowing for easy access, has been met by securing the external portion approximate to exit site of the catheter using a gauze wrapping. In the case of a PICC line, this is done by coiling the external portion, holding it against the skin of the patient's arm, and wrapping some length of gauze or cloth mesh around the patient's arm such that the external portion of the PICC is held between the patient's arm and the length of gauze or mesh. Although such method is inexpensive and instantly customizable to all sizes of patients, it has two significant drawbacks. First, it is quite uncomfortable for the plastic pieces of the PICC line to be held so tightly against the skin of a patient. Often, these pieces leave an irritating imprint in the skin of the patient after the wrapping has been removed. Second, the wrapping is aesthetically displeasing. This is a significant drawback, as a PICC may remain inserted for a number of months, during which time the patient is often free to go about his or her normal daily activities in and out of public.

Therefore, the goals of the present invention are to provide an inexpensive, customizable and aesthetically pleasing device that may comfortably retain the external portion of an infusion device, such as a PICC line.

### SUMMARY

The present invention provides an inexpensive, customizable and aesthetically pleasing device that may comfortably retain the external portion of an infusion device, such as a PICC line. The invention essentially involves a band of comfortable and visually pleasing material adapted to be worn approximate to the location of the external portion of the infusion device, where the band includes an outer pocket adapted to receive and safely secure the external portion of the device when not in use. The invention also includes a method for temporarily storing the external portion of an infusion device within the pocket of the aesthetically pleasing and comfortable band when worn by the patient.

A first exemplary embodiment of the present invention includes the following elements: 1) a circular band, having an inner surface and an outer surface; 2) a pocket facing attached to the outer surface of the circular band forming a pocket that is adapted to retain an external portion of the PICC line; and 3) a retainer for retaining the circular band around a patient's arm. The circular band and pocket facing may be constructed from a sheet material, preferably a washable sheet material. In a more detailed embodiment, the circular band may be constructed from two or more layers of sheet material and the retainer could be an elastic band that is visually concealed among the two or more lawyers of sheet material.

A second exemplary embodiment of the present invention includes the following elements: 1) a strip of sheet material having a first lateral edge, a second lateral edge, a first end, and a second end; 2) a first fastener attached to the first end of the strip of sheet material and a corresponding second fastener attached to the second end of the strip of sheet material, such that the strip of sheet material becomes a circular band, having an inner surface and an outer surface, when the first fastener engages the corresponding second fastener; and 3) a pocket facing attached to the outer surface between the first end and the second end forming a pocket that is adapted to retain an external portion of the PICC line. This exemplary embodiment may also be constructed from washable sheet material. In a more detailed embodiment, the first fastener and the corresponding second fastener could be a mating pair of hook and loop fasteners, such as Velcro® brand hook and loop fasteners. Such an embodiment could allow a user to alter the circumference of the circular band by altering a point of engagement between the first fastener and the second corresponding fastener. Alternatively, the first fastener could be one or more hooks and the second fastener could be one or more eyes. Further, the second fastener could include two or more rows of one or more eyes, where each row is located at a different distance from the second end of the strip of sheet material. Using this embodiment, a user could alter the circumference of the circular band by altering the row of one or more eyes that is engaged by the one or more hooks. Regardless of the type of fastener chosen, the circular band could also be constructed from two or more layers of sheet material; and a biasing mechanism could be used to retain the circular band around the patient's arm. Such biasing mechanism could be an elastic band that is visually concealed among the two or more layers of sheet material.

Any embodiment of the present invention could further provide one or more graphic elements displayed on the outer surface of the circular band or on the pocket facing. Such graphic elements could include: 1) an amusing character; 2) a visually interesting design; 3) a name of a healthcare system or institution; or 4) a logo of a healthcare system or institution.

### BRIEF DESCRIPTION OF THE DRAWINGS

FIGS. 1 and 2 are diagrams of a patient wearing a PICC line and an exemplary embodiment of the present invention.

FIG. 3 shows four pieces of sheet material.

FIG. 4 shows a first step in the method of making an exemplary embodiment of the present invention.

FIG. 5 shows a second step in the method of making an exemplary embodiment of the present invention.

FIG. 6 is a flow chart of a third step in the method of making an exemplary embodiment of the present invention.

FIG. 7 shows a fourth step in the method of making an exemplary embodiment of the present invention.

FIGS. 8 and 9 show alternative fifth steps in the method of making two alternative exemplary embodiments of the present invention.

### DETAILED DESCRIPTION

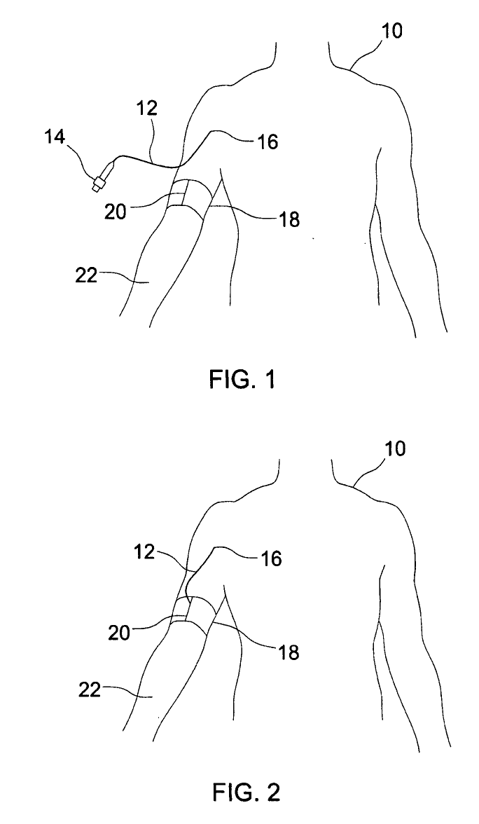

The present in invention is generally directed to a device for storing on the patient's body the external portion of an infusion device. For exemplary purposes only, an embodiment of the present invention adapted for use with a PICC line is herein described in detail. FIG. 1 depicts a patient 10 wearing a PICC line 12. The PICC line enters a vein in the patient's arm 22 at the exit site 16, and is threaded through the vein to a termination point under the patient's clavicle, close to the patient's heart. The external portion of the PICC line 12 includes some length of tubing terminating in a PRN adapter 14. When the PICC line 12 is in use, the PRN adapter 14 connects the PICC line 12 to any number of devices used to deliver medications or other fluids. When the PICC line 12 is not in use, the external tubing and PRN adapter 14 is disconnected from such devices and thus, without some means for support, would hang free from the exit site 16 along the patient's arm 22 (as shown in FIG. 1). If the external portion of the PICC line is permitted to hang free without support, it could easily be disturbed, causing any number of uncomfortable and/or medically dangerous conditions.

The traditional method of supporting the external tubing and PRN adapter 14 while not in use is to wrap some length of fabric gauze around the patient's arm and the external portion of the PICC line, thereby holding the external portion tightly against the patient's arm. This method is both uncomfortable for the patient and unsightly. The present invention was thus developed to eliminate these disadvantages to the traditional method, while maintaining the necessary support function. To promote support, comfort and aesthetics, while remaining inexpensive to manufacture, the present invention was developed to comprise an arm band 18, to be worn on the arm 22 of a patient 10, with a pocket 20, for storing the external portion of a PICC line 12 when not in use. FIG. 2 depicts a patient 10 utilizing the present invention to store the external portion of a PICC line 12.

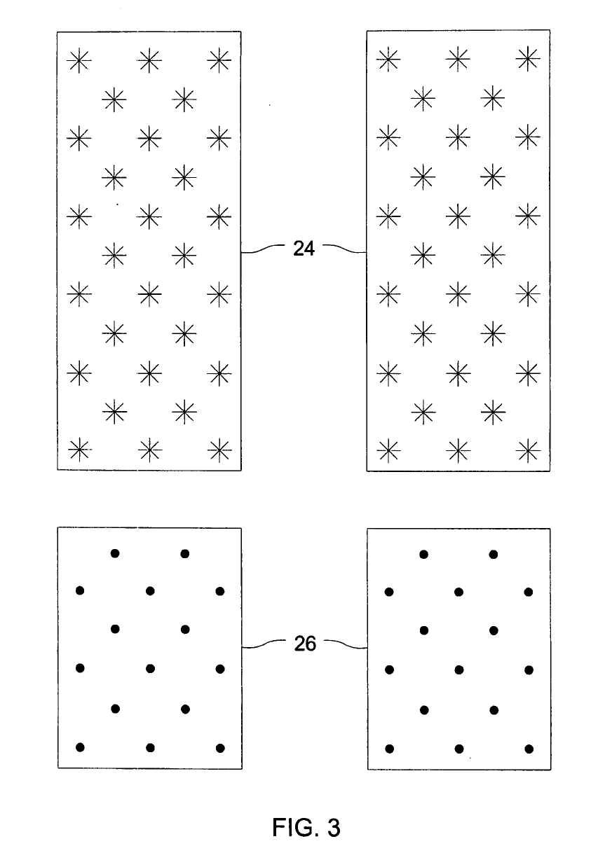

The exemplary embodiments of the present invention are constructed from four pieces of sheet (fabric) material, two larger rectangular pieces 24 and two smaller rectangular pieces 26, as shown in FIG. 3. In a more detailed exemplary embodiment, the larger rectangular pieces 24 are twelve inches long and four inches wide, and the smaller rectangular pieces 26 are five and one half inches long and four inches wide. The sheet material may be a washable sheet material, facilitating easy cleaning of the device, and/or may depict one or more visual designs. Examples of such visual designs include (without limitation): any visual design that is interesting or aesthetically pleasing to the patient; or a hospital or other organizational name or logo. An amusing character, such as an animal or cartoon character, could be depicted for use by a pediatric patient.

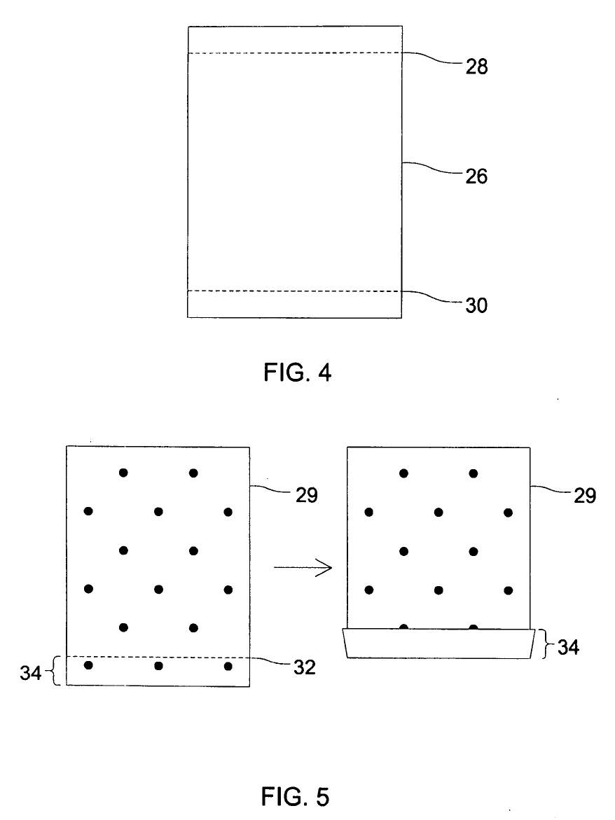

A first step in constructing an exemplary embodiment of the present invention from the four pieces of sheet material is to place the two smaller rectangular pieces 26, one on top of the other, as depicted in FIG. 4. If only one side of the fabric is printed, or the fabric otherwise has a front side and a back side, the smaller rectangular pieces 26 should be stacked so that the printed or front sides are facing in toward each other. Lines 28 and 30 are then sewn across the two shorter sides of the stack; the fabric is turned inside-out; and the new seams are pressed. The resulting double-layer of fabric is the pocket facing 29 for the pocket portion of the exemplary embodiment.

The second step in constructing an exemplary embodiment of the present invention is to measure a line 32 that is five eighths of an inch from one of the new seams and fold a portion both layers of material 34 such that the crease is along line 32, as depicted in FIG. 5. This fold should also be pressed, so that the crease remains along line 32 when the fabric is unfolded.

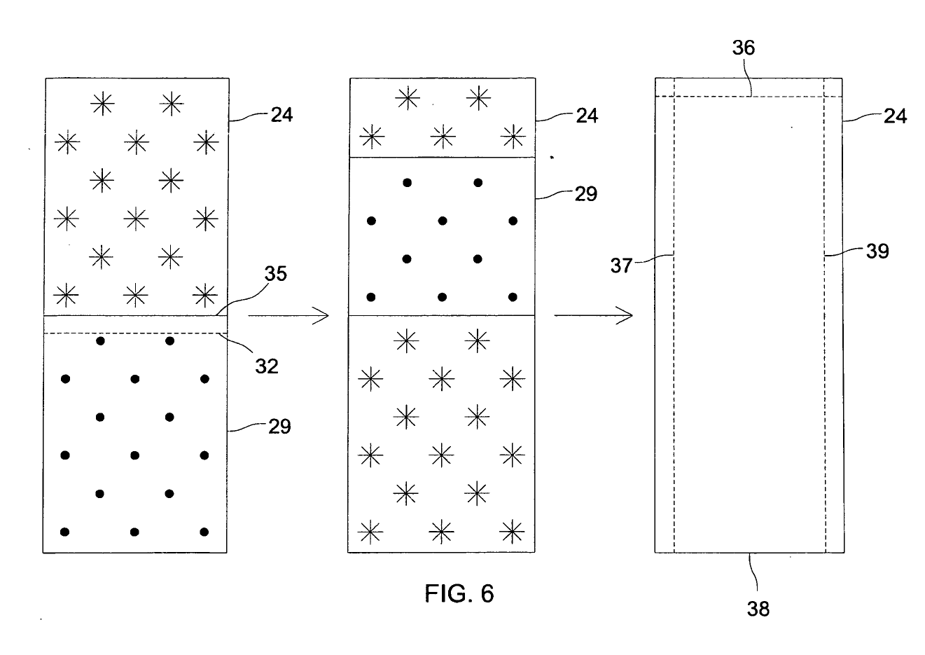

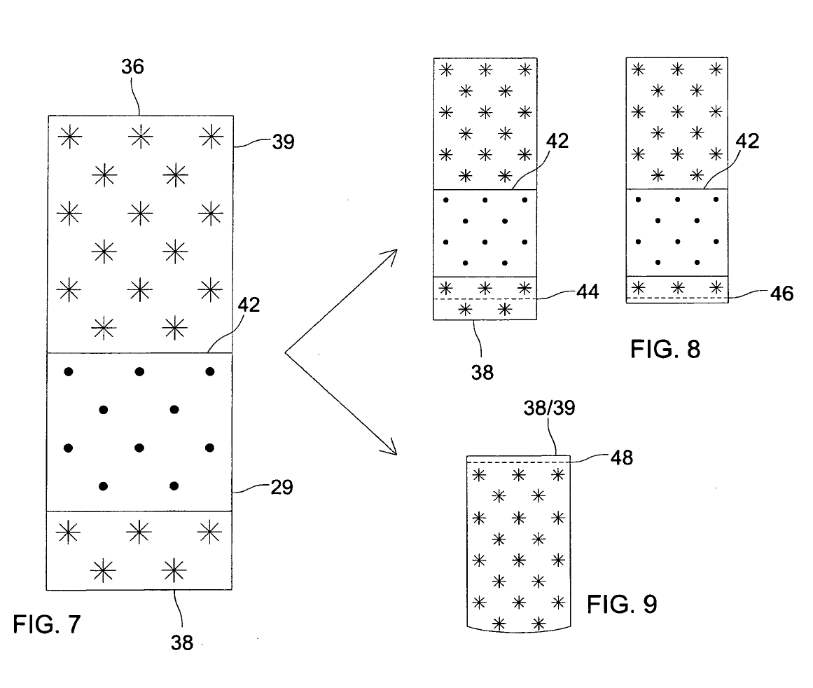

The third step in constructing an exemplary embodiment of the present invention, as shown in FIG. 6, is to place one of the larger rectangular sheets of material 24 with the printed or front side facing up. A line 35 is then measured seven inches down from the top edge of the sheet 24. Then the pocket facing 29 is placed on top of the sheet of material 24 such that the seam edge adjacent to line 32 abuts line 35, as shown in FIG. 6. A line is then sewn across line 32 and the pocket facing 29 is flipped upward, creasing at line 32. The second larger rectangular sheet of material 24 is then placed on top, print or front side down, and lines 36, 37 and 39 are sewn along three of the edges of the entire stack of material, as shown in FIG. 6. A person skilled in the art would appreciate that an elastic band could be inserted along the length of the assembly or a series of hooks and eyes or corresponding strips of hook and loop fasteners, such as Velcrog strips, could be placed on opposite ends 36 and 38 of the assembly, before lines 36, 37 and 39 are sewn. After lines 36, 37 and 39 are sewn, the open end 38 of the assembly is used to turn the assembly inside-out, resulting in a band similar to that shown in FIG. 7.

At this point, there are two finishing options. If fasteners such as hooks and eyes or corresponding strips of hook and loop fasteners have been used, the band should be finished in a linear configuration, as shown in FIG. 8. To do this, a line 44 is drawn five eighths of an inch from the open end 38. Then the raw edges of material at open end 38 are folded inward at line 44 and a line 46 is sewn along the folded edge. If this option is chosen, the band can be placed around a patient's arm, pocket side facing outward, and secured in the circular configuration by engaging the fasteners at either end of the band. To remove the band, the fasteners are simply disengaged and the band returns to its linear configuration.

If fasteners have not been attached to the ends of the band, the band can be finished such that it is in a permanent circular configuration. This finishing option, as shown in FIG. 9 is accomplished by loosely folding the band in half such that ends 38 and 39 meet with the pocket 29 on the inside of the fold; sewing a line 48 along the ends; and turning the entire band inside-out. Because the exemplary embodiment constructed by this method must be pulled on and off the patient's arm in its circular configuration, it is best if an elastic band is sewn into it to provide for some flexibility and a secure fit.

Once the present invention has been placed around the arm of a patient, it is used by simply coiling any external portion of the patient's PICC line and placing that external portion in the pocket of the present invention.

Any of these exemplary embodiments may be easily adapted by size and circumference to secure other types of infusion devices, such as those having exit sites on a patient's leg or abdomen. For example, an embodiment of slightly larger circumference could be worn around a patient's leg to secure the external portion of a femoral catheter. Likewise, an embodiment composed of a wider band of even greater circumference could be worn around the abdomen of a patient to secure the external portion of an insulin infusion device, such as an insulin pump. Each of these embodiments provides the desired comfort, aesthetics and support. Further, it would be apparent to a person skilled in the art that such a device would be inexpensive to manufacture.

Having described the invention by reference to exemplary embodiments of the invention, it will be apparent to those of ordinary skill that it is possible to fall within the scope of the invention as defined, at least in part, by the following claims without necessarily practicing the exemplary embodiments of the invention. Likewise, it will be apparent to those of ordinary skill in the art that changes can be made to the exemplary embodiments of the invention without departing from the scope of the invention as defined in the following claims.

What is claimed is:

1. A storage and support device for retaining an external portion of an infusion device, the storage and support device comprising:
a circular band having an inner surface and an outer surface;
a pocket facing attached to the outer surface of the circular band to form a pocket that is adapted to retain an external portion of an infusion device; and
a retainer for retaining the circular band on a patient's body approximate to an exit site of the infusion device.

2. The storage and support device of claim 1, wherein the circular band and pocket facing are composed of a sheet material.

3. The storage and support device of claim 1, wherein the circular band and pocket facing are composed of a washable sheet material.

4. The storage and support device of claim 2, wherein at least a portion of the sheet material displays one or more graphic elements from a listing including:
an amusing character;
a visually interesting design;
a name of a healthcare system or institution; and
a logo of a healthcare system or institution.

5. The storage and support device of claim 2, wherein the circular band is constructed from two or more layers of the sheet material.

6. The storage and support device of claim 5, wherein the retainer includes an elastic band that is visually concealed among the two or more lawyers of sheet material.

7. The storage and support device of claim 6, wherein the infusion device is a peripherally inserted central catheter, and the storage and support device is adapted to be worn around an arm of the patient.

8. A storage and support device for retaining an external portion of an infusion device, the storage and support device comprising:
a strip of sheet material having a first lateral edge, a second lateral edge, a first end, and a second end;
a first fastener attached to the first end of the strip of sheet material and a corresponding second fastener attached to the second end of the strip of sheet material, such that the strip of sheet material becomes a circular band, having an inner surface and an outer surface, when the first fastener engages the corresponding second fastener; and
a pocket facing attached to the outer surface between the first end and the second end to form a pocket that is adapted to retain an external portion of an infusion device.

9. The storage and support device of claim 8, wherein the strip of sheet material is composed of a washable sheet material.

10. The storage and support device of claim 9, wherein at least a portion of the sheet material displays one or more graphic elements from a list including:
an amusing character;
a visually interesting design;
a name of a healthcare system or institution; and
a logo of a healthcare system or institution.

11. The storage and support device of claim 8, wherein the first fastener and the corresponding second fastener are a mating pair of hook and loop fasteners.

12. The storage and support device of claim 11, wherein a circumference of the circular band may be changed by changing a point of engagement between the first fastener and the corresponding second fastener.

13. The storage and support device of claim 12, wherein the infusion device is a peripherally inserted central catheter, and the storage and support device is adapted to be worn around the arm of the patient.

14. The storage and support device of claim 8, wherein the first fastener is one or more hooks and the corresponding second fastener is one or more eyes.

15. The storage and support device of claim 14, wherein the second fastener includes a series of rows of one or more eyes, each row positioned a different distance from the second end of the strip of sheet material, such that a circumference of the circular band may be changed by changing the row of one or more eyes engaged by the one or more hooks.

16. The storage and support device of claim 15, wherein the infusion device is a peripherally inserted central catheter, and the storage and support device is adapted to be worn around the arm of the patient.

17. The storage and support device of claim 15, wherein the strip of sheet material includes of two or more layers of sheet material.

18. The storage and support device of claim 17, further comprising a biasing mechanism for retaining the circular band around a patient's arm.

19. The storage and support device of claim 18, wherein the biasing mechanism includes an elastic band that is visually concealed among the two or more layers of sheet material.

20. The storage and support device of claim 19, wherein the infusion device is a peripherally inserted central catheter, and the storage and support device is adapted to be worn around the arm of the patient.

21. A band of comfortable and visually pleasing material adapted to be worn on the patient's arm, approximate to a location of an external portion of a catheter line extending from the patient's arm, where the band includes an outer pocket adapted to receive and safely secure the external portion of the catheter line when not in use.

22. A method for temporarily storing within a pocket of an aesthetically pleasing and comfortable band, an external portion of an infusion device extending from a patient's body, wherein the band is worn by the patient approximate to an exit site of the infusion device.

23. A method for temporarily storing an external portion of a catheter line extending from a patient's arm within a pocket of an aesthetically pleasing and comfortable arm-band worn by the patient.
